(19) United States
(12) Patent Application Publication
(10) Pub. No.: US 2012/0088941 A1
(43) Pub. Date: Apr. 12, 2012

(54) POLYOL REFINING

(75) Inventors: Hans-Karl Soest, Cologne (DE); Ulrich Litzinger, Hachenburg (DE); Reinhold Klipper, Cologne (DE); Rudolf Wagner, Cologne (DE)

(73) Assignee: LANXESS DEUTSCHLAND GMBH, Leverkusen (DE)

(21) Appl. No.: 13/329,932

(22) Filed: Dec. 19, 2011

Related U.S. Application Data
(63) Continuation of application No. 12/027,355, filed on Feb. 7, 2008, now abandoned.

(30) Foreign Application Priority Data
Jul. 25, 2007 (DE) 10 2007 034621.4

(51) Int. Cl. C07C 31/22; C07C 31/18

(52) U.S. Cl. 568/870; 568/868

(57) ABSTRACT

The subject of the present invention is a method for refining of polyols, preferably glycerol, by means of monodispersed ion exchangers in a purification unit of ion exclusion process and a mixed bed.

This application is a continuation of U.S. patent application Ser. No. 12/027,355 filed Feb. 7, 2008, pending, which claims the benefit to German patent application No. 10 2007 034621.4, filed Jul. 25, 2007, incorporated herein by reference.

The subject of the present invention is a method of polyol refining, preferably for glycerol, by means of monodispersed ion exchanger in a purification unit consisting of an ion exclusion process and a mixed bed.

### BACKGROUND OF THE INVENTION

Due to the increased synthesis of biodiesel from renewable raw materials in recent time, considerable quantities of glycerol are accruing, which is loaded with a considerable percentage of ions, especially sodium and chloride, and may have a deep brown discoloration. And both of these factors are undesirable for the further processing of glycerol, for example, into cosmetics, in the food industry, or into pharmaceutical products.

Purifying glycerol by means of ion exchangers is known in the prior art. U.S. Pat. No. 7,126,032 B1 describes the purification of glycerol from biodiesel fabrication, including with ion exchangers. Likewise, numerous product brochures of renowned makers of ion exchangers recommend the use of ion exchangers for the desalting of glycerol, such as the Dow Chemical Company Dowex HCR-W2, a strong-acid, gel-like cation exchanger (16-40 mesh), or Lanxess Deutschland GmbH under the brand name Lewatit the ion exchangers S1428, S1468, S2528, S2568, S3428, S4228, S4268, S4328, S6328, S6368 or MDS1368Natrium.

Not always does the use of the mentioned ion exchangers achieve the purities of the polyol, especially glycerol, required for particular branches of industry. Therefore, there is a desire to obtain polyols from fatty acid alkyl ester processes, preferably glycerin, in such a purity that it/they fulfil the high demands of the cosmetics industry, the food industry, or the pharmaceutical industry for their raw material.

### SUMMARY OF THE INVENTION

The solution of the problem and thus the object of the present invention is a method for refining of polyols, characterized in that one uses a purification unit made up of an ion exclusion process and a mixed bed. In a preferred embodiment, at least one monodispersed ion exchanger is used in this purification unit.

The ion exclusion process, hereinafter IEC (ion exclusion chromatography), is a known prior art. It is used, for example, for the fractionation of silage juice into an amino acid and a lactic acid fraction.

The term used in the older German literature for ion exclusion chromatography is electrolyte first run process. This describes the primary separation mechanism quite well. Electrolytes (inorganic ions, organic ions) are excluded from the ion exchange matrix and the entire electrolyte fraction passes through the chromatography column as if it were filled with glass beads. For example, IEC is also used to separate sugars (WO 2003056038 A1) or to get ethanol (WO 1995017517 A1).

Many ion exchangers are available for use in IEC, including monodispersed ion exchangers. Thus we find under http://www.dow.com/liquidseps/prod/chromato.htm the monodispersed Dowex Monosphere 99K 320 for use in amino acid production or the production of organic acids, as well as for production of sugar from sugar beets or sugar cane. In the present invention, IEC is used as a method for desalting of polyol, preferably glycerol.

A mixed bed, or mixed bed resins, are a mixture of at least one strongly acidic cation exchanger and a strongly basic anion exchanger, optimally attuned to each other. These resins also easily remove “difficult” contents of water, such as silicic acid and carbonic acid. They are preferably used for total desalination of water. For example, mixed beds are described in US 20050103622 A1 and especially in U.S. Pat. No. 5,858,191, and the latter in particular is subsumed in its entirety by the present patent in this respect.

Unlike heterodispersed ion exchangers with heterodispersed particle size distribution, which one obtains by traditional methods, the present application uses the term monodispersed to mean ion exchangers in which at least 90 vol. or wt. % of the particles have a diameter which lies in the interval around the most frequent diameter with width of ±10% of the most frequent diameter.

For example, for an ion exchanger with most frequent bead diameter of 0.5 mm, at least 90 vol. or wt. % lie in a size interval between 0.45 mm and 0.55 mm; for a substance with most frequent diameter of 0.7 mm, at least 90 vol. or wt. % lie in a size interval between 0.77 mm and 0.63 mm.

A monodispersed bead polymerizate required for the production of monodispersed ion exchangers can be produced according to the methods known from the literature. For example, such methods and the monodispersed ion exchangers made from them are described in U.S. Pat. No. 4,444,961, EP-A 0 046 535, U.S. Pat. No. 4,419,245 or WO 93/12167, whose contents are fully subsumed by the present application. According to the invention, monodispersed bead polymerizates and the monodispersed ion exchangers prepared from them are obtained by jetting or seed/feed processes. Preferably, according to the invention, at least one monodispersed ion exchanger is contained in the IEC or in the mixed bed. In an especially preferred embodiment, one monodispersed ion exchanger is contained in each of the IEC and the mixed bed. Very preferred according to the invention, only monodispersed ion exchangers are contained in the IEC as well as the mixed bed.

Preferably according to the invention, strong-acid cation exchangers are used in the IEC, especially preferably strong-acid, gel-like cation exchangers. Especially preferably according to the invention, monodispersed, strong-acid, gel-like cation exchangers are used, such as Lewatit GF 303.

The polyol which is largely salt-free obtained after the treatment in the IEC, especially glycerol, is subjected to a fine cleaning in a mixed bed in the second stage according to the invention, aiming for a salt content of less than 1 ppm. In addition, one achieves a so-called polishing of the polyol in the mixed bed, whereby a very low color value of almost entirely clear is achieved. For this, preferably an anion exchanger and a cation exchanger are used alongside each other. Especially preferably, one of the resins used in the mixed bed is monodispersed, especially preferably, both ion exchangers in the mixed bed are monodispersed.

The terms microporous, macroporous or gel-like have already been described fully in the technical literature. Preferred anion exchangers or cation exchangers in the mixed bed have a macroporous structure.

The formation of macroporous bead polymerizates for the production of macroporous ion exchangers can take place, for example, by adding inert materials (pore-forming agents) to the monomer mixture during the polymerization. Suitable as such are first and foremost organic substances that dissolve in the monomer, but dissolve or swell the polymerizate slightly (precipitating agents for polymers), such as aliphatic hydrocarbons (Farbenfabriken Bayer DBP 1045102, 1957; DBP 1113570, 1957).

The pore-forming agents used in U.S. Pat. No. 4,382,124 are alcohols with 4 to 10 carbon atoms for preparation of monodispersed, macroporous bead polymerizates on a styrene/divinyl benzene basis. Moreover, a survey is given as to the methods of production of macroporous bead polymerizates. Preferable as pore-forming agents according to the invention are organic solvents which poorly dissolve or swell the resulting polymerizate. Preferred pore-forming agents are hexane, octane, isooctane, isododecane, methylethylketone, butanol or octanol or their isomers.

Therefore, the combination of a monodispersed macroporous cation exchanger with a monodispersed, macroporous anion exchanger is preferred in the mixed bed according to the invention, and a monodispersed, macroporous, strong-acid cation exchanger with a monodispersed, macroporous, medium-basic anion exchanger is especially preferred. As an example of a mixed bed, one can mention here Lewatit GF 404 in combination with Lewatit GF 505.

Surprisingly, by the method of the invention, namely, by means of a purification unit of IEC and a mixed bed, one achieves polyols in such high purity and such outstanding color values that they can be used with no further processing in the cosmetic industry, the food industry, or the pharmaceutical industry. In the case of glycerol, salt contents of less than 1 ppm and color values of less than 1 IU (International Unit; Sugar Analysis, Icumsa Methods, F. Schneider, 1979, Paragraph 7, Physical Characteristics of Colour of Sugar and Solutions) are achieved.

But the present invention also concerns the use of at least one, preferably two, most preferably at least three monodispersed ion exchangers inside a purification unit consisting of IEC and mixed bed for the refining of polyols, especially glycerol.

Moreover, the present invention concerns the use of a purification unit consisting of IEC and mixed bed in the production of biodiesel for the processing of the polyol accruing during the production, preferably glycerol. The invention moreover concerns a method for production of biodesel, characterized in that the polyol feedstock is subjected to a purification unit consisting of IEC and a mixed bed. In a preferred embodiment, the method is characterized by

a) the transesterification of free fatty acids into fatty acid esters by means of macroporous cation exchanger,

b) the separation of the biodiesel from the polyol and

c1) the processing of the polyol by means of a purification unit made up of IEC and mixed bed, and

c2) the processing of the biodiesel to remove the polyol and/or soaps by a strong-acid, monodispersed, macroporous cation exchanger.

In an especially preferred embodiment, a heretodispersed, macroporous, highly sulfonated cation exchanger is used in step a) and a strong-acid, monodispersed, gel-like cation exchanger in step c2). For step a), one can mention here Lewatit GF 101 and for step c2) Lewatit GF 303 or Lewatit K 2567 from Lanxess Deutschland GmbH.

It will be understood that the specification and examples are illustrative but not limitative of the present invention and that other embodiments within the spirit and scope of the invention will suggest themselves to those skilled in the art.

### EXAMPLES

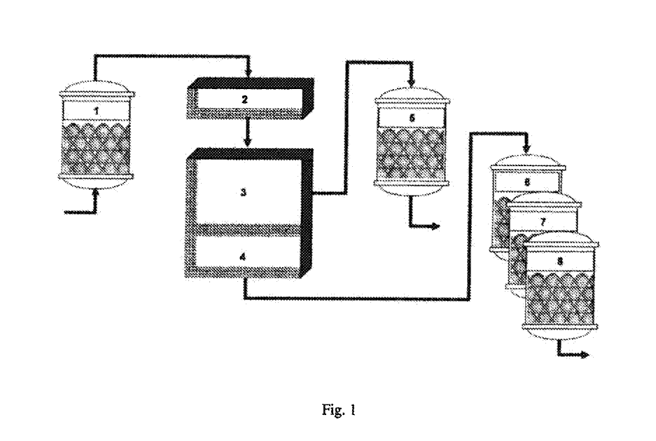

FIG. 1 shows schematically a production plant for biodiesel with subsequent cleaning of the biodiesel as well as the accruing polyol, in this case, glycerol.

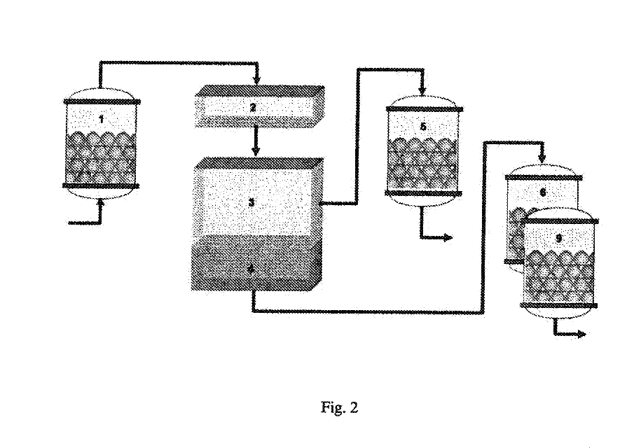

FIG. 2 likewise shows schematically a production plant for biodiesel with the difference that, contrary to a connection of a mixed bed, as in FIG. 1, a single apparatus contains the mixed bed here.

Position 1 in FIG. 1 stands for an apparatus that is filled with an esterification catalyst in order to separate fatty acids from the triglycerides. Of course, natural oils, such as rapeseed oil, consist of a mixture of triglycerides (>95%), fatty acids (0.1 to 5%), and micelles, phospholipids, proteins and mineral salts (<1%).

Preferably, an esterification catalyst of type Lewatit GF 101 or Lewatit K 2620 or Lewatit K 2621 is used in 1, or in the case of an enzymatic esterification Lewatit GF 808 or Lewatit OC 1600.

The transesterification process takes place in 2, being followed by the separation of the two phases, the biodiesel phase 3 from the glycerol phase 4.

The biodiesel phase goes through an apparatus 5 filled, for example, with a monodispersed strong acid macroporous cation exchanger of type Lewatit K 2567 or Lewatit GF 202 or Lewatit SP112 for the removal of residual glycerol, soaps, waxes, salts, water or methanol.

The glycerol phase goes through a purification unit according to the invention, made up of apparatuses 6 as well as 7 and 8, or alternatively 9 (FIG. 2), in which 6 stands for the IEC and 7 and 8 for a connection of apparatuses of a mixed bed and 9 (FIG. 2) stands for an individual apparatus as mixed bed. In 6, according to the invention, a mondispersed gel-like strong-acid cation exchanger is used to separate salts or ash from the glycerol, such as Lewatit GF 303.

In 7, for example, a monodispersed, macroporous, strong-acid cation exchange is used as polisher, and also to remove cations, such as Lewatit GF 404. In 8, preferably a monodispersed, macroporous, medium-basic anion exchanger is used as polisher and also to separate anions, but also to decolorize the glycerol, such as Lewatit GF 505.

In 9 (FIG. 2), for example, a monodispersed, macroporous, strong-acid cation exchanger is used as polisher, and also to separate cations, such as Lewatit GF 404, and a monodispersed, macroporous, medium-basic anion exchanger, such as Lewatit GF 505 or a monodispersed, strong-basic anion exchanger of type I or type II, for example, Lewatit S 6368 A or Lewatit S 7468 is used to separate anions, but also to decolorize the glycerol in a mixture in a volumetric ratio of cation exchanger 1:anion exchanger 0.8 to 2. The difference between anion exchangers of type I and type II is described, for example, in Ullmann's Encyclopedia of Technical Chemistry, Verlag Chemie, Weinheim, New York, 4^{th} ed., Vol. 13, p. 302.

The cation exchanger in apparatus 7, after the existing exchanger capacity is used up, is regenerated by means of diluted mineral acids, preferably 4-10 wt. % hydrochloric acid, sulfuric acid, or nitric acid. The regenerating solution can be filtered either from the top or from the bottom through the ion exchanger. After this, the regenerating solution is expelled with deionized water while maintaining the direction of filtration. After this comes a washing with deionized water in the outflow until the pH value at the exit from the apparatus is 5-6.

The anion exchanger in apparatus 8, after the existing exchanger capacity is used up, is regenerated by means of diluted lye, preferably 3-8 wt. % sodium hydroxide. The regenerating solution can be filtered either from the top or from the bottom through the ion exchanger. After this, the regenerating solution is expelled with deionized water while maintaining the direction of filtration. After this comes a washing with deionized water in the outflow until the pH value at the exit from the apparatus is 7-8.

The components of the resin mixture (cation exchanger and anion exchanger) in apparatus 9, after the exchanger capacity is used up, are first separated by back-flushing with deionized water and then the individual resins are individually regenerated. The anion exchanger is regenerated with NaOH (3-6 wt. %) from the top and the cation exchanger with an aqueous solution of HCl, preferably up to 5-8 wt. %, from the bottom simultaneously. The regeneration solutions are taken off by a drainage situated at the height of the resin separation zone. After this, the regeneration solutions are expelled and rinsing is done with deionized water in the direction of the respective chemical solutions.

TABLE 1
Example for the design of a 10,000 ton per year IEC plant of a
fixed bed for glycerol ash and salt removal per FIG. 1,
position 3 with Lewatit GF 303 as the resin used

| | Glycerol from biodiesel |
|---|---|
| Medium being purified | transesterification |
| Resin volume | 30 m3 |
| Diameter of resin bed | 2.5 m |
| Depth of resin bed | 6.0 m |
| Charge per cycle | 3.75 T of glycerol in |
| | 23 T of deionized water |
| Salt concentration of the crude | 5-7 wt. % |
| glycerol | |
| Eluate | Deionized water |
| Temperature | 80 degrees C. |
| Outflow | 3.75 T of glycerol in |
| | 6 T of deionized water |
| Lifetime of resin | 5 years |

Deionized water in the sense of the present invention is characterized in that it has a conductivity of 0.1 to 10 μS, and the content of dissolved or undissolved metal ions is not greater than 1 ppm, preferably not greater than 0.5 ppm for Fe, Co, Ni, Mo, Cr, Cu as individual components and not greater than 10 ppm, preferably not greater than 1 ppm, for the total of said metals.

TABLE 2
Example for the design of a 10,000 ton per year
glycerol polishing mixed bed unit

| | |
|---|---|
| Resin quantity in 7 | 5 m3 of Lewatit GF 404 |
| Resin quantity in 8 | 6 m3 of Lewatit GF 505 |
| NaCl concentration before 7 | 100 ppm |
| Color value of the glycerol before 7 | 200 IU |
| Flow rate through 7 and 8 | 20 m3/h |
| Temperature | 60 degrees C. |
| Capacity of the mixed bed | 3000 m3/cycle |
| Cycle time | 150 h |
| NaCl concentration after 8 | <1 ppm |
| Color value of the glycerol after 8 | <1 IU |

The information about the glycerol was measured with a UV/VIS spectral photometer of type CADAS 30 S from the Dr. Lange firm, Berlin. The information on the color values of the glycerol in the context of the present invention is therefore referred to measurements with such an instrument, while:

IU=1000×Ext_{(420 mm)×}100/b×c×D

Where Ext=−log transmission, b=dry substance in ⁰bx, c=cell length in cm and D=density.

### Preparation of Lewatit GF 303 for the IEC

### Lewatit GF 303 is a Gel-Like, Monodispersed, Strong Acid Cation Exchanger in the Sodium Form

### a) Preparation of the Monodispersed, Gel-Like Bead Polymerizate

985.6 grams of an aqueous mixture containing 492.8 grams of monodispersed microencapsulated monomer droplets with a mean particle size of 230μ and a degree of monodispersion of 1.11, consisting of 93.5 wt. % of styrene, 6 wt. % of divinyl benzene, and 0.5 wt. % of dibenzoyl peroxide, were reacted with an aqueous solution of 1.48 grams of gelatin, 2.22 grams of sodium hydrogen phosphate dodecahydrate and 110 mg of resorcin in 40 ml of deionized water in a 4 liter glass reactor.

The mixture was polymerized under stirring (stirring speed 220 rpm) for 6 hours at 70 degrees C. and then for 2 hours at 95 degrees C. The batch was washed using a 32μ screen and dried. One gets 512 grams of a monodispersed, gel-like bead polymerizate, bead diameter 275μ, with smooth surface.

b) Sulfonation of the Monodispersed, Gel-Like Bead Polymerizate into a Monodispersed, Gel-Like Cation Exchanger and Conversion of the Cation Exchanger from the Hydrogen Form to the Sodium Form

Apparatus: 3000 ml double-wall planar ground reactor with intensive cooler, agitator and drying pistol

2241 g of 85 wt. % sulfuric acid at room temperature was placed in the vessel. Under agitation, 400 grams of monodispersed, gel-like bead polymerizate was added over 5 minutes. Then, 150 ml of 1,2-dichlorethane was added. The suspension was agitated at room temperature for 3 hours. Over the course of 1 hour, 829.8 grams of 65% oleum was added. The suspension was heated to 120 degrees C. over the course of 1 hour and agitated at this temperature for another 4 hours. Dichlorethane was driven off by distillation.

The suspension was cooled down to room temperature and transferred to a dilution apparatus, where it was diluted with sulfuric acid of decreasing concentration.

The resin cooled down to room temperature was washed with deionized water and then classified.

After this, 4400 ml of 4 wt. % aqueous sodium hydroxide was filtered across the resin for 2 hours and then 3000 ml of deionized water was filtered across the resin.

Yield of end product: 2010 ml

Total capacity: quantity of strong acid groups: 1.92 mol/l

### Preparation of Lewatit GF 404 for the Mixed Bed

### Lewatit GF 404 is a Macroporous, Monodispersed, Strong-Acid Cation Exchanger in the Hydrogen Form

### A′) Preparation of a Monodispersed, Macroporous Bead Polymerizate Based on Styrene, Divinyl Benzene and Ethyl Styrene

In a 10 liter glass reactor, 3000 g of deionized water was placed, and a solution of 10 g of gelatin, 16 g of disodium hydrogen phosphate dodecahydrate and 0.73 g of resorcin in 320 g of deionized water was added and mixed with it. The mixture was tempered at 25 degrees C. While stirring, a mixture of 3200 g of microencapsulated monomer droplets with narrow particle size distribution of 8.5 wt. % divinyl benzene and 2.1 wt. % ethyl styrene (used as an off-the-shelf isomer mixture of divinyl benzene and ethyl styrene with 80% divinyl benzene), 0.5 wt. % Trigonox 21 s, 56.5 wt. % of styrene and 32.4 wt. % of isododecane (technical-grade isomer mixture with high fraction of pentamethyl heptane) was added, while the microcapsule consisted of a formaldehyde-hardened complex coacervate of gelatin and a copolymer of acrylamide and acrylic acid, and 3200 g of aqueous phase with a pH value of 12 was added. The mean particle size of the monomer droplets was 460 μm.

The batch was polymerized under agitation by raising the temperature by a temperature program starting at 25 degrees C. and ending at 95 degrees C. The batch was cooled down, washed through a 32 μm sieve and then dried in vacuum at 80 degrees C. One gets 1893 g of a ball-shaped, macroporous polymerizate with a mean particle size of 440 μm, narrow particle size distribution, and smooth surface.

The bead polymerizate was chalk-white in appearance.

B′) Sulfonation of the Monodispersed, Macroporous Bead Polymerizate into a Monodispersed, Macroporous Cation Exchanger in the Hydrogen Form

Apparatus: 3000 ml double-wall planar ground reactor with intensive cooler, agitator and drying pistol

1000 ml of 98 wt. % of sulfuric acid at room temperature was placed in the vessel and heated to 105 degrees C. Under agitation, 250 grams of monodispersed, macroporous bead polymerizate was added over 30 minutes. The suspension was then heated to 115 degrees C. over the course of 1 hour and agitated at this temperature for another 5 hours.

The suspension was cooled down to room temperature and transferred to a dilution apparatus, where it was diluted with sulfuric acid of decreasing concentration.

The resin cooled down to room temperature was washed with deionized water and then classified.

Yield of end product: 1225 ml

Total capacity: quantity of strong acid groups: 1.61 mol/l

### Preparation of Lewatit GF 505 for the Mixed Bed

### Lewatit GF 505 is a Macroporous, Monodispersed, Medium-Basic Anion Exchanger

### A″) Preparation of a Monodispersed, Macroporous Bead Polymerizate Based on Styrene, Divinyl Benzene and Ethyl Styrene

In a 10 liter glass reactor, 3000 g of deionized water was placed, and a solution of 10 g of gelatin, 16 g of disodium hydrogen phosphate dodecahydrate and 0.73 g of resorcin in 320 g of deionized water was added and mixed with it. The mixture was tempered at 25 degrees C. While stirring, a mixture of 3200 g of microencapsulated monomer droplets with narrow particle size distribution of 3.6 wt. % divinyl benzene and 0.9 wt. % ethyl styrene (used as an off-the-shelf isomer mixture of divinyl benzene and ethyl styrene with 80% divinyl benzene), 0.5 wt. % Trigonox 21 s, 56.2 wt. % of styrene and 38.8 wt. % of isododecane (technical-grade isomer mixture with high fraction of pentamethyl heptane) was added, while the microcapsule consisted of a formaldehyde-hardened complex coacervate of gelatin and a copolymer of acrylamide and acrylic acid, and 3200 g of aqueous phase with a pH value of 12 was added. The mean particle size of the monomer droplets was 460 μm.

The batch was polymerized under agitation by raising the temperature by a temperature program starting at 25 degrees C. and ending at 95 degrees C. The batch was cooled down, washed through a 32 μm sieve and then dried in vacuum at 80 degrees C. One gets 1893 g of a ball-shaped, macroporous polymerizate with a mean particle size of 440 μm, narrow particle size distribution, and smooth surface.

The bead polymerizate was chalk-white in appearance and had a bulk density of around 370 g/l.

### B″) Preparation of an Amidomethylated Bead Polymerizate

At room temperature, 1856.3 ml of dichlorethane, 503.5 g of phthalimide and 351 g of 29.9 wt. % formalin were placed in a vessel. The pH value of the suspension was adjusted with sodium hydroxide to 5.5 to 6. The water was then driven off by distillation. Then 36.9 g of sulfuric acid was added. The resulting water was driven off by distillation. The batch was cooled down. At 30 degrees C., 134.9 g of 65% oleum and then 265.3 g of monodispersed bead polymerizate, prepared by step A″), was added. The suspension was heated to 70 degrees C. and agitated at this temperature for another 6 hours. The reaction liquor was decanted, deionized water was added, and residual quantities of dichlorethane were driven off by distillation.

Yield of amidomethylated bead polymerizate: 1700 ml

Elemental analysis composition:

Carbon: 75.1 wt. %;

Hydrogen: 4.7 wt. %;

Nitrogen: 5.8 wt. %;

Rest: oxygen

### C″) Preparation of an Aminomethylated Bead Polymerizate

To 1680 ml of amidomethylated bead polymerizate from B″), 773.3 g of 50 wt. % sodium hydroxide and 1511 ml of deionized water at room temperature was added. The suspension was heated to 180 degrees C. over the space of 2 hours and agitated at this temperature for 8 hours. The obtained bead polymerizate was washed with deionized water.

Yield of aminomethylated bead polymerizate: 1330 ml

Elemental analysis composition:

Nitrogen: 11.6 wt. %;

Carbon: 78.3 wt. %;

Hydrogen: 8.4 wt. %;

From the elemental analysis composition, of the aminomethylated bead polymerizate one can calculate that 1.18 hydrogen atoms have been replaced by aminomethyl groups in the statistical mean per aromatic unit—deriving from the styrene and divinylbenzene units.

Determination of the quantity of basic groups: 2.17 mol/liter of resin

D″) Preparation of a Bead Polymerizate with Tertiary Anion Groups

In a reactor, 1875 ml of deionized water, 1250 ml of aminomethylated bead polymerizate from C″) and 596.8 g of 30.0 wt. % formalin solution at room temperature was placed. The suspension was heated to 40 degrees C. The pH value of the suspension was adjusted to pH 3 by adding 85 wt. % formic acid. The suspension was heated to reflux temperature (97 degrees C.) within the course of 2 hours. During this time, the pH value was held at 3.0 by adding formic acid. After reaching the reflux temperature, the pH value was adjusted to 2 at first by adding formic acid and then by adding 50 wt. % of sulfuric acid. Additional stirring was done at pH 2 for 30 minutes. Then, more 50 wt. % sulfuric acid was added and the pH value adjusted to 1. Stirring was done for another 8.5 hours at pH 1 and reflux temperature.

The batch was cooled down, the resin filtered off on a screen and washed with deionized water.

Volume yield: 2100 m.

In a column, filtration was done across the resin with 4 wt. % aqueous sodium hydroxide. Washing was then done with water.

Volume yield: 1450 ml

Elemental analysis composition:

Determination of the quantity of basic groups: 1.79 mol/liter of resin

### E″) Preparation of a Monodispersed, Medium-Strong Basic Anion Exchanger

In a reactor at room temperature, 700 ml of anion exchanger with tertiary amino groups from example D″), 780 ml of deionized water and 16.5 grams of chlormethane were placed. The batch was heated to 40 degrees C. and agitated at this temperature for 6 hours.

Volume yield: 951 ml

Of the nitrogen-carrying groups of the anion exchanger, 24.3% were present as trimethylaminomethyl groups and 75.7% as dimethylaminomethyl groups.

What is claimed is:

1. A method for refining a polyol mixture, said polyol mixture comprising a polyol and non-polyol compounds, comprising the steps of:
contacting said polyol mixture with a first ion exchanger, whereby ion exclusion chromatography (IEC) is performed and thereby forming a second polyol mixture in which a portion of the non-polyol compounds have been removed; and
contacting said second polyol mixture with a second ion exchanger, said second ion exchanger being m the form of a mixed bed ion exchanger thereby forming a third polyol mixture in which a portion of the non-polyol compounds of the second polyol mixture have been removed.

2. The method according to claim 1, wherein the first ion exchanger is a monodisperse ion exchanger.

3. The method according to claim 2, wherein the monodisperse ion exchanger is formed of monodispersed bead polymerizates via a jetting or seed/feed process.

4. The method according to claim 1, wherein the second ion exchanger is a monodisperse ion exchanger.

5. The method according to claim 4, wherein the monodisperse ion exchanger is formed of monodispersed bead polymerizates via a jetting or seed/feed process.

6. The method according to claim 1, wherein the first and the second ion exchangers are each monodisperse ion exchangers.

7. The method according to claim 6, wherein the monodisperse ion exchangers are formed of monodispersed bead polymerizates via a jetting or seed/feed process.

8. The method according to claim 1, wherein the first ion exchanger is a strong acid cation exchanger.

9. The method according to claim 8, wherein the strong acid cation exchanger is a gel-like strong acid cation exchanger.

10. The method according to claim 1, wherein the second ion exchanger comprises both cation and anion exchange resin.

11. The method according to claim 10, wherein either or both of the cation and anion exchange resin are monodispersed.

12. The method according to claim 1, wherein the polyol is glycerol.

13. The method according to claim 12, wherein the polyol mixture is a biodiesel production derivative.

14. A method for producing biodiesel, wherein a polyol feedstock is purified by means of a purification unit, said purification unit comprising an ion exchanger capable of ion exclusion chromatography (IEC) and a mixed bed ion exchanger.

15. A method for producing biodiesel comprising:
esterifying a free fatty acid into fatty acid esters in the presence of a strongly acidic, macroporous cation exchanger,
transesterifying at least one triglyceride into further fatty acid esters, whereby polyols are formed as a byproduct of the transesterifying, thereby forming a mixture comprising said further fatty acid esters and polyols;
separating the further fatty acid esters from the polyols, thereby forming a first polyol mixture; and
purifying the first polyol mixture via a purification unit, wherein
a first ion exchanger processes the first polyol mixture by ion exclusion chromatography, said first ion exchanger being in the form of a strong-acid, monodispersed, gel-like cation exchanger, and
a mixed bed ion exchanger comprising one or more further ion exchangers, further processes the polyol mixture by ion exchange separation.

16. The method according to claim 15, wherein the mixed bed ion exchanger is a single apparatus.

17. The method according to claim 15, wherein the mixed bed ion exchanger is formed of multiple apparatuses.

18. The method according to claims 15, further comprising:
regenerating the mixed bed ion exchanger by contacting the same with dilute mineral acids or lyes.
